(19) United States
(12) Patent Application Publication
(10) Pub. No.: US 2014/0228807 A1
(43) Pub. Date: Aug. 14, 2014

(54) Intravenous Catheter Timing Device

(71) Applicant: Glennah D. Hart, Columbia, SC (US)

(72) Inventor: Glennah D. Hart, Columbia, SC (US)

(21) Appl. No.: 13/765,562

(22) Filed: Feb. 12, 2013

(51) Int. Cl. A61M 5/50

(52) U.S. Cl. CPC A61M 5/5086
USPC 604/508; 604/111

(57) ABSTRACT

The invention designed as a means for indication that an in-use intravenous catheter is within prescribed safe time limits for the prevention of healthcare acquired infections. The device consists of an indicator light and timer integrated with an angiocath.

### CROSS REFERENCE TO RELATED APPLICATIONS

Not Applicable.

### STATEMENT REGARDING FEDERALLY SPONSORED RESEARCH OR DEVELOPMENT

This invention was not made by an agency of the United States Government nor under a contract with an agency of the United States Government.

### PARTIES TO JOINT RESEARCH AGREEMENT

Not Applicable.

### REFERENCE TO SEQUENCE LISTING

Not Applicable.

### BACKGROUND OF THE INVENTION

A nosocomial infection, also known as a hospital-acquire infection or healthcare-associated infection (HAI), are infections that patients acquire during the course of receiving healthcare treatment. The United States Center for Disease Control (CDC) estimates that 1 out of every 20 hospitalized patients will contract an HAI. Annual cost of treatment of HAIs is in the tens of billions of dollars in the United States alone. Approximately two-thirds of HAIs are associated with the use of catheters and ventilators. Other HAIs include infections at surgical sites and gastrointestinal infections resulting from exposure to contaminated surfaces or unclean hands of others.

Hospitals and other health care facilities have implemented many measures designed to prevent HAIs. Among them are hygiene standards, use of protective equipment such as disposable gloves, sterilization of equipment, use of skin antisepsis before breaking the skin for surgical procedures or intravenous catheter (iv) insertions, and time limits for iv catheters insertion sites.

Patients can also develop a condition known as phlebitis of the vein when an iv catheter must be used for extended periods. Phlebitis may cause inflammation or swelling at the iv site and can lead to the formation of blood clots. Changing the iv location is a preventative measure against phlebitis. This invention was conceived and design to aid health care providers in the prevention of phlebitis and hospital acquired infections in patients who require treatment via intravenous catheter.

### BRIEF SUMMARY OF THE INVENTION

The invention described in this application is a relatively low cost means for indication that an in-use intravenous catheter is within prescribed safe time limits for the prevention of healthcare acquired infections. The device consists of an indicator light (light emitting diode or LED) and timer integrated with an angiocath. The indicator light and timer are located on the outside of the angiocath so that the sterile environment of the angiocath interior is maintained and that the timer LED indication is readily visible to health care personnel and the patient. Prior to inserting the needle portion of the angiocath into a patient's vein, an activation tag on the timer indicator subsystem is pulled to enable the timer. The LED indicator on the angiocath will glow green. The angiocath can then be utilized in the normal manner. After a factory preset time has elapsed (typically 72 or 96 hours), the indicator light will turn from green to red, thus highlighting that the iv has been in use beyond the time limits directed by hospital policy and needs to be replaced.

### BRIEF DESCRIPTION OF THE SEVERAL VIEWS OF THE DRAWING

FIG. 1 shows a top perspective of the invention.

FIG. 2 shows the invention as inserted into a patient's vein.

FIG. 3 shows the LEDs, timer and protective case.

### DETAILED DESCRIPTION OF THE INVENTION

The invention described in this application is designed to assist hospitals and other health care facilities in their efforts to prevent nosocomial infections, also known as a hospital-acquire infections or healthcare-associated infections (HAI). These infections acquire by patients during the course of receiving healthcare treatment. The United States Center for Disease Control (CDC) estimates that 1 out of every 20 hospitalized patients will contract an HAI. Annual cost of treatment of HAIs is in the tens of billions of dollars in the United States alone. Approximately two-thirds of HAIs are associated with the use of catheters and ventilators. The invention covered by this application is designed to assist prevent infections and phlebitis of the veins that can result from the use of catheters, in particular intravenous catheters or IVs.

A standard IV infusion set consists of a pre-filled, sterile container of fluids with an attachment that allows the fluid to flow one drop at a time, a long sterile tube with a clamp to regulate or stop the flow; a connector to attach to the access device; and connectors (called “Y-tubes or Y-Set (infusion))” to allow “piggybacking” of another infusion set onto the same line, e.g., adding a dose of antibiotics to a continuous fluid drip. An angiocath is inserted into a patient's vein and is connected to the tubing to allow flow of the fluid from the external source into the patient's vein. Gravity is the simplest motive force for the fluid flow and is achieved by locating the sterile container of fluid at a higher elevation than the patient. An infusion pump may also be used to allow more precise control over the flow rate of the liquid.

Any break in the skin carries the risk of infection. Bacteria can enter the body around the insertion site of the iv catheter, moisture from washing or bathing can increase the infection risk. In an effort to prevent infections at IV insertion sites, many hospitals implemented policies that limit the amount of time the IV can be left at a particular insertion point unless the patient has poor venous access. The CDC guidelines state that there is no need to replace IV catheters more frequently than 72 to 96 hours.

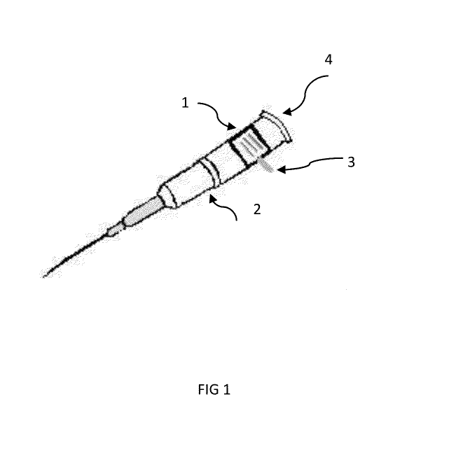

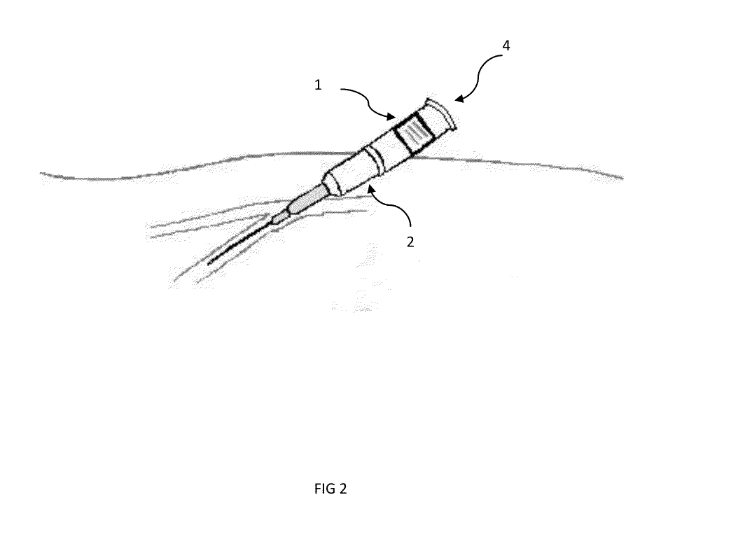

The invention described in this application is a relatively low cost means for indication that an in-use intravenous catheter is within prescribed safe time limits for the prevention of healthcare acquired infections. As seen in FIG. 1, the device consists of an indicator light and timer (1) integrated with an angiocath (2). The indicator light and timer are located on the outside of the angiocath so that the sterile environment of the angiocath interior is maintained and that the timer LED indication is readily visible to health care personnel, the patient and the patient's family. The LED indicator and timer are powered by an integrated battery requiring no external electrical connections. The timer and indicator are activated by the removal of a strip (3) by the health care provider prior to inserting the angiocath into a patient's vein (FIG. 2). The LED indicator, timer and battery do not come into contact with the fluid being conveyed via the iv tubing (4), thus maintaining a sterile environment. The device is an inexpensive one-time use instrument that will require minimal training of personnel to use.

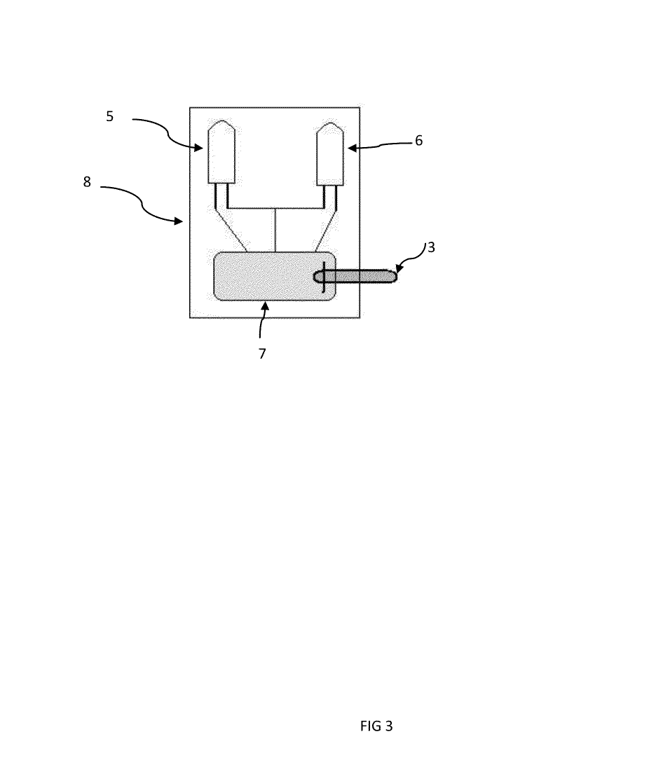

As seen in FIG. 3, the indicator/timing device attached to the angiocath is comprised of a translucent container (8) inside of which is a green colored light emitting diode (5), a red colored light emitting diode (6), a microchip timer/power source (7) and an activation strip (3). The entirety of this device (1) is attached to an angiocath (2) as seen in FIG. 1.

Prior to inserting the needle portion of the angiocath into a patient's vein, an activation tag on the timer indicator subsystem is pulled to enable the timer and the green LED. The angiocath can then be utilized in the normal manner. After a factory preset time has elapsed (typically 72 or 96 hours), the timer microchip will turn off the green LED and turn on the red LED, thus highlighting that the angiocath has been in use beyond the time limits directed by hospital policy and needs to be replaced.

The invention described in this application differs significantly from United States Patent Application Publication Number 2011/0004153—Catheter Time Monitoring System and Methods of Using The Same, inventor Anita Kipping (published Jan. 6, 2011). The Kipping invention is a device that can provide time in service for the overall fluid catheter system, but not the specific components of the catheter system. The invention described in this application is specifically designed to indicate the safe service life of the portion of an iv catheter that is inserted into the patient and thus carries the greatest risk of infection.

The Kipping invention, as described in its various embodiments, appears to be a device designed for repeated utilization. When used for delivery of sterile fluids via an intravenous catheter, the unit would require sterilization as there is direct contact with the sterile fluid. If it is a repeated use device, the Kipping invention could become a source of infection unless proper sterilization procedures are followed between usages. The invention covered by this application is designed to be a one-time use device provided in sterile packaging from the manufacturer.

As noted in the paragraph above, the Kipping invention appears to be desired for repeated utilization. Repeated usage would require the timing device to be reset for each new use. The ability to reset the timer would introduce an element of unreliability should the timer be mistakenly or inadvertently reset while in use. In contrast, once activated the timer portion of the invention covered by this application cannot be reset and potentially extend the time the angiocath was in the patient beyond the specified safe zone.

The invention described in this application differs significantly from United States Patent Application Publication Number 2011/0009817—Intravenous Fluid Monitoring, inventors James W. Bennett and Leonid F. Matsiev (published Jan. 13, 2011). The Intravenous Fluid Monitoring system is designed to monitor the identity of the fluid between injected into the patient via the intravenous catheter and not the time the iv catheter is in use.

The invention described in this application differs significantly from United States Patent Application Publication Number 2010/0309005—Automated Intravenous Monitoring Device, inventors Tod. H. Warner and Wayne Carmen (published Dec. 9, 2010). The Automated Intravenous Monitoring Device is designed to monitor the flow rate and quantity of the fluid between injected into the patient via the intravenous catheter and not the time the iv catheter is in use.

A potential patent classification for this invention is Class 604: Surgery; Sub-Class 164.11: Cannula.

The present invention described above and shown in the accompanying drawings is visualized as the preferred embodiment of the invention. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a stricture or feature that is disposed “adjacent” another feature may have portions that overlap or underlie the adjacent feature.

The invention claimed is:

1. An intravenous catheter timing device comprising a timing subsystem, an activation strip, a power source and two different colored Light Emitting Diodes (LED) inside a translucent case which is attached to an angiocath.

2. The device according to claim 1, wherein said timing subsystem is activated by removal of said activation strip.

3. The device according to claim 1, wherein said LED indicator of color one is activated when the timer is activated.

4. The device according to claim 1, wherein said timing subsystem after a preset period of time, of about 72 hours, deactivated said LED indicator of color one and activates said LED indicator of color two.

5. The device according to claim 1, wherein said LED indicator color one is green.

6. The device according to claim 1, wherein said LED indicator color two is red.

7. A method of preventing hospital acquired infections and phlebitis at the site of an intravenous iv catheter by utilizing a device comprising a timer and LED indicator capable of displaying at least two different colors to indicate whether the intravenous iv has been in use within the prescribed safe zone period of time or whether the usage has extended beyond the safe zone period of time.
